(19) United States
(12) Patent Application Publication
(10) Pub. No.: US 2020/0254221 A1
(43) Pub. Date: Aug. 13, 2020

(54) MINIMALLY INVASIVE SURGICAL DRAIN IMMOBILIZATION SYSTEM AND METHOD

(71) Applicant: Joshua Burkin, Daytona Beach, FL (US)

(72) Inventor: Joshua Burkin, Daytona Beach, FL (US)

(21) Appl. No.: 16/786,622

(22) Filed: Feb. 10, 2020

Related U.S. Application Data
(63) Continuation-in-part of application No. 16/590,346, filed on Oct. 1, 2019.
(60) Provisional application No. 62/803,793, filed on Feb. 11, 2019.

(51) Int. Cl. A61M 25/02; A61M 27/00

(52) U.S. Cl. CPC A61M 25/02; A61M 27/00; A61M 2205/0205; A61M 2025/0266; A61M 2025/024

(57) ABSTRACT

A drain tube immobilization device includes a clasp having a top body section and a bottom body section that are pivotally connected via a hinge along a first end. The clasp transitions between an open position and a closed position, and is secured in the closed position by a locking mechanism. Each of the body sections include a plurality of complimentary-oriented semicircular recesses, that form individual channels when the clasp is in the closed and locked position. Each of the channels including a shape and size that is complementary to the cross-sectional shape and size of a surgical drain tube. A malleable base member is permanently secured along the bottom of the clasp, and medical grade skin adhesive is positioned along the bottom surface of the base member.

### CROSS-REFERENCE TO RELATED APPLICATIONS

This application is a continuation-in-part to, and claims the benefit of U.S. application Ser. No. 16/590,346 filed on Oct. 1, 2019, and U.S. Application Ser. No. 62/803,793 filed on Feb. 11, 2019, the contents of each of which are incorporated herein by reference.

### TECHNICAL FIELD

The present invention relates generally to medical devices, and more particularly to a surgical drain securement device.

### BACKGROUND

The statements in this section merely provide background information related to the present disclosure and may not constitute prior art.

Over the years, millions of surgical drains have been placed in patients worldwide. A surgical drain allows pus, blood and other fluids from a wound or other area to flow out of a patient's body. To accomplish this, a doctor will position the first end of a thin (typically latex) drain tube into the area of the body where fluid is likely to collect. The drain tube will then carry the fluid outside the body through a small incision wherein a squeeze bulb or other apparatus is attached to the second end of the tube. The surgical drain uses suction created by the bulb to pull the fluid from the body into the bulb. Depending on the type of surgery performed, or if the patient has multiple injuries, the shape, size and/or number of drain tubes may vary greatly.

In either instance, to prevent the drain tube(s) from accidentally being pulled from the wound site, it is common practice to secure a portion of each drain tube onto the patient's body at a location adjacent to the wound site. Current medical standards teach doctors to do this by physically sewing the drain tube to the patient's skin via sutures or stitches, for example.

This method has several drawbacks, in that it is painful to the patient, causes trauma to the suture site, often leaves a permanent scar, and ultimately requires a doctor, or other surgical personnel, to remove the sutures from the patient. Moreover, with each additional drain tube that is used, the number of painful stitches increases, and so does the risk for secondary infections.

Accordingly, it would be beneficial to provide a multi-channel drain tube immobilization device that is capable of engaging multiple drain tubes of different shapes and sizes, that does not suffer from the drawbacks described above.

### SUMMARY OF THE INVENTION

The present invention is directed to a multi-channel drain tube immobilization device. One embodiment of the present invention can include a clasp having a top body section and a bottom body section that are pivotally connected via a hinge along a first end. The clasp can transition between an open position and a closed position, and can be secured in the closed position by a locking mechanism. Each of the body sections can include a plurality of complimentary-oriented semicircular recesses, that form individual channels when the clasp is in the closed and locked position. Each of the channels including a shape and size that is complementary to the cross-sectional shape and size of a surgical drain tube.

Another embodiment of the present invention can include a malleable base member that is permanently secured along the bottom of the multi-channel clasp. The base member can include medical grade skin adhesive along the bottom surface, and can function to secure the clasp to a patient's body.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of an exemplary drain tube immobilization device in the open position, in accordance with one embodiment of the invention.

FIG. 2 is a side view of an exemplary drain tube immobilization device in the closed position, in accordance with one embodiment of the invention.

FIG. 3 is a perspective view of the exemplary drain tube immobilization device in operation, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

Although illustrated for use with a surgical drain line, the inventive concepts are not to be construed as limited for use with any particular type of line. As such, embodiments are contemplated wherein the below described device can be sized to engage and secure a variety of objects to a body such as IV lines, chest tubes, feeding tubes and the like, for example.

### Definitions

As described herein, the term “removably secured,” and derivatives thereof shall be used to describe a situation wherein two or more objects are joined together in a non-permanent manner so as to allow the same objects to be repeatedly joined and separated.

As described herein, the term “pivotally secured” “rotatably secured” and derivatives thereof shall be used interchangeably to describe a situation wherein two identified objects are joined together in a manner that allows one or both of the objects to pivot, rotate, swing or otherwise move about or in relation to the other object in a horizontal or vertical manner. Several nonlimiting examples of connectors for pivotally connecting objects together include traditional single hinge mechanisms, ball joint couplers, and/or swivel flanges, for example.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

As described herein, the term “connector” can include any number of different elements that work to join two items together. Several nonlimiting examples include opposing strips of hook and loop material (i.e. Velcro®), attractively-oriented magnetic elements, flexible strips of interlocking projections with a slider (i.e., zipper), tethers, buckles, and compression fittings such as draw latches, hooks, snaps and buttons, for example. Each illustrated connector can be permanently secured to the illustrated portion of the device via a permanent sealer such as glue, adhesive tape, or stitching, for example.

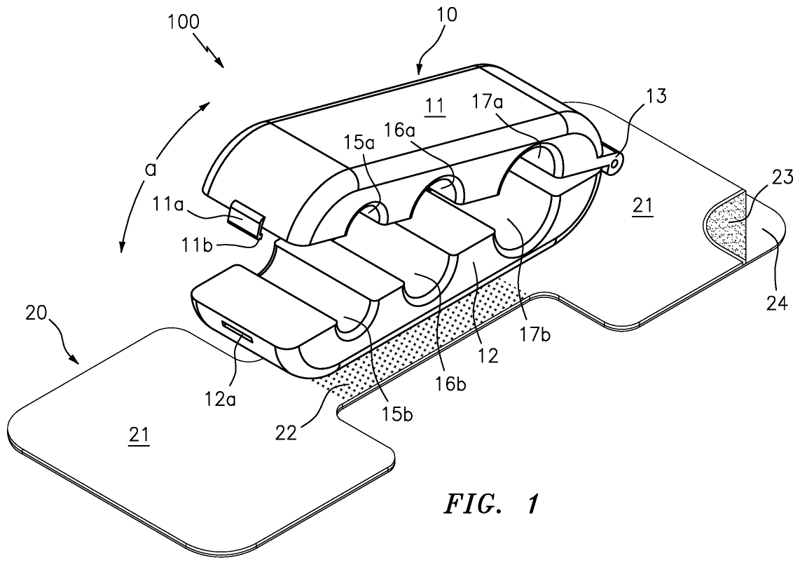

FIGS. 1-3 illustrate one embodiment of a drain tube immobilization device 100 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

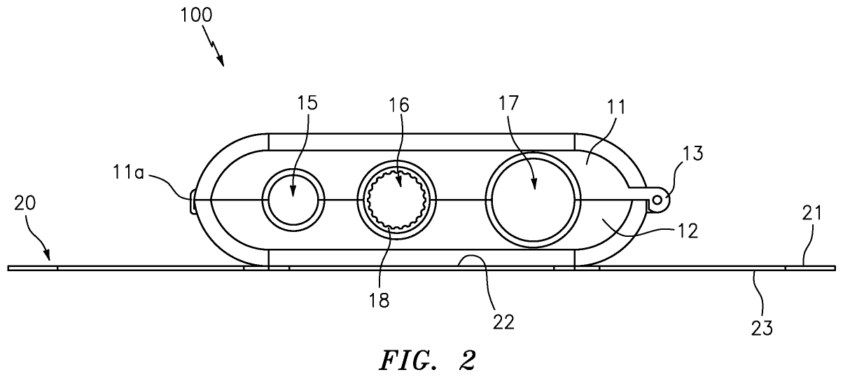

As shown at FIGS. 1 and 2, the drain tube immobilization device 100 can include, essentially, a multi-channel surgical drain clasp 10, and a base member 20.

In one embodiment, the clasp 10 can be constructed from anti-microbial plastic, and can include a top body section 11 and a bottom body section 12. In the preferred embodiment, the top and bottom body sections 11 and 12 may be pivotally secured together along one end via a hinge 13 or other such component capable of allowing the top body section to transition between an open and closed position, see arrow a.

In one embodiment, the clasp can include a locking mechanism whereby the top body section includes an actuator 11a having a protrusion 11b that extend downward therefrom. The bottom body section 12 can include a recess 12a located along one surface. The actuator functioning to selectively position the protrusion within the recess to lock/secure the clasp 10 in the closed position shown at FIG. 2.

Although described above as including a hinge and specific locking mechanism components, this is for illustrative purposes only. To this end, any number of other components capable of removably securing the top and bottom body sections together so as to facilitate use of the device in the manner hereinafter described are also contemplated.

As shown, both the top body section 11 and the bottom body section 12 can each include a plurality of semicircular recesses 15a, 16a, 17a, and 15b, 15b and 17b, respectively. In one embodiment, recesses 15a and 15b can include identical shapes and sizes to each other, recesses 16a and 16b can include identical shapes and sizes to each other, and recesses 17a and 17b can include identical shapes and sizes to each other. Of course, other embodiments are contemplated wherein one or more of the above identified recesses includes a different shape and/or size than another recess.

As shown, each of the recesses can preferably be positioned perpendicular to the major axis of the top and bottom body sections, and can be located at complementary locations so as to be aligned when the clasp is in the closed position. In this regard, each of the semicircular recess 15a-15b, 16a-16b, and 17a-17b can form one half of an individual channel 15, 16 and 17, respectively.

In one embodiment, each of the individual channels 15, 16 and 17 can each be sized to include an inside diameter that is complementary to the outside diameter of a commercially available surgical drain line. As is known to those of skill in the art, most commercially available surgical drain lines have an outside diameter of either 4 cm, 7 cm and 10 cm, therefore, these sizes are preferred.

In various embodiments, the clasp 10 may be constructed wherein channels 15, 16 and 17 may each include identical sizes to each other, so as to allow a single device 100 to receive and engage multiple drain lines of the same size/diameter. Likewise, the clasp 10 may be constructed wherein channels 15, 16 and/or 17 may include different sizes to each other, so as to allow a single device 100 to receive and engage drain lines having any number of different sizes/diameters. Moreover, although illustrated as including three cylindrical-shaped channels, other embodiments are contemplated wherein a single clasp 10 can include more channels or fewer channels, each having any number of different shapes and/or sizes.

In various embodiments, one or more of the semicircular recesses can include a nonskid lining 18. The lining will preferably include or consist of a rubber material that is soft (e.g., does not have protrusions) so as to not puncture a drain line, but that also increases the friction of the device to prevent slippage of an inserted drain line. Of course, any number of other materials are also contemplated.

The base member 20 can function to secure the multi-channel clasp 10 onto a patient's body at any desired location. In one nonlimiting embodiment, the base member 20 can include a body section 21 that is constructed from a durable and malleable material such as thin (e.g., 1 mm-2 mm) vinyl, for example.

In the preferred embodiment, the bottom surface of the clasp 10 can be permanently secured onto the top surface of the body member 21 via a permanent adhesive 22 such as acrylic resin or other such materials that create a strong permanent bond between two items.

In the preferred embodiment, the bottom surface of the body section 21 can be coated with medical grade skin adhesive 23, for example, and can be covered by a releasable liner 24 until ready for use. Of course, any number of other adhesives and/or connectors capable of removably securing the body section onto the skin of a user are also contemplated.

Although described above as utilizing various types of adhesives, this is for illustrative purposes only, as any number of other substances and/or connectors capable of performing the stated functionality are also contemplated.

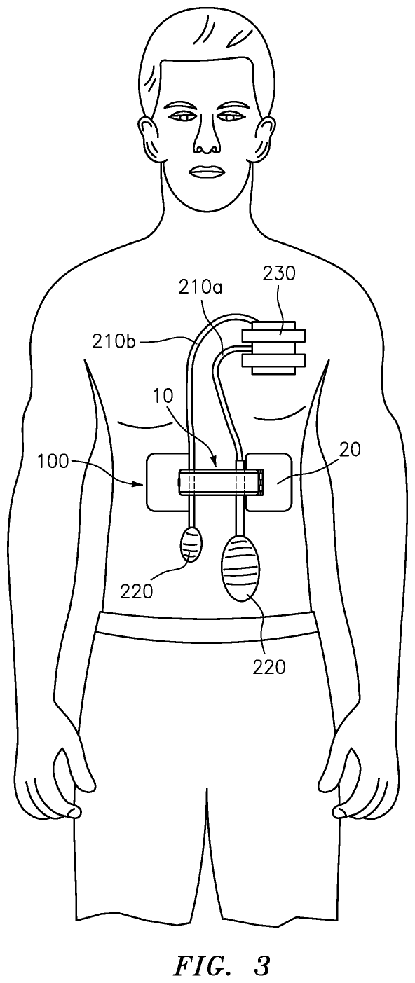

FIG. 3 illustrates one embodiment of the drain tube immobilization device 100 in operation. As shown, any number of drain lines/tubes 210a and 210b can extend from a patient's body 230 before terminating at a bulb 220 or other medical instrument. As such, the base member 20 can first be positioned onto the skin of the patient with the multi-channel clasp in the open position. Next, each of the lines 210a-210b can be positioned within one of the semicircular channels of the clasp, and the device can be transitioned to the closed position, thus immobilizing the lines against the patient's body.

When the lines are no longer needed, they can be removed from the device 100 by reversing the above noted steps, and the device can then be discarded.

Accordingly, the above noted drain tube immobilization device 100 advantageously functions to removably secure any number of different shaped and sized drain lines and other such devices to a patient's body in a pain free manner that does not require stitches or result in permanent disfigurement of the patient's body.

As described herein, one or more elements of the drain tube immobilization device 100 can be secured together utilizing any number of known attachment means such as, for example, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the terms “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A drain tube immobilization device, comprising:
a clasp having a top body section with a first plurality of semicircular recesses, and a bottom body section with a second plurality of semicircular recesses; and
a base member having a top surface that is in communication with the clasp, and a bottom surface having an adhesive material disposed thereon,
wherein the clasp includes functionality for transitioning between an open position and a closed position.

2. The device of claim 1, wherein the first plurality of semicircular recesses and the second plurality of semicircular recesses are aligned to form a plurality of individual channels when the clasp is in the closed position.

3. The device of claim 2, wherein the plurality of individual channels are positioned perpendicular to a major axis of the clasp.

4. The device of claim 2, wherein each of the plurality of individual channels include an identical shape and an identical size.

5. The device of claim 2, wherein at least one of the plurality of individual channels includes a shape that is different than a shape of another of the plurality of individual channels.

6. The device of claim 2, wherein at least one of the plurality of individual channels includes a size that is different than a size of another of the plurality of individual channels.

7. The device of claim 1, wherein the top body section is pivotally connected to the bottom body section along a first end of the clasp by a hinge.

8. The device of claim 7, further comprising:
a locking mechanism that is disposed along a second end of the clasp.

9. The device of claim 8, wherein the locking mechanism comprises:
an actuator having a protrusion that extends downward from the top body section; and
a recess that is disposed along the bottom body section.

10. The device of claim 1, further comprising:
a removable lining that is in communication with the adhesive material along the bottom surface of the base member.

11. The device of claim 1, wherein the adhesive material comprises medical grade adhesive glue.

12. The device of claim 1, wherein the clasp is constructed from antimicrobial plastic.
